(19) United States
(12) Patent Application Publication
(10) Pub. No.: US 2025/0270264 A1
(43) Pub. Date: Aug. 28, 2025

(54) Radiodurans-Suscepta-Humana_01_2009_rev_01_02_2024

(71) Applicant: Henry Makowitz, Idaho Falls, ID (US)

(72) Inventor: Henry Makowitz, Idaho Falls, ID (US)

(73) Assignee: Imagineering Technologies, Incorporated, Idaho Falls, ID (US)

(21) Appl. No.: 18/831,357

(22) Filed: Dec. 12, 2024

Related U.S. Application Data
(60) Provisional application No. 63/630,703, filed on Feb. 26, 2024.

(51) Int. Cl. C07K 14/195; A61K 35/74

(52) U.S. Cl. CPC C07K 14/195; A61K 35/74

(57) ABSTRACT

There exist in NATURE a number of microscopic organisms that exhibit survival traits under extreme conditions. Several exhibit survival traits under extensive radiation fields. One such organism is Deinococcus Radiodurans. (refer to Refs 1 a and 1 b of PRIOR ART) We believe that this survival trait is genetically based. We claim that this genetic trait can be utilized for enhancement of radiation susceptibility and resistance and can be encapsulated in a pharmaceutical. This is partially confirmed in Ref 1c for a human cell system (refer to the PRIOR ART section), and justifies our Patent. We claim as our invention a pharmaceutical selectively utilizing the Deinococcus Radiodurans genes, and DNA/RNA genetic as well as chemical repair system, for the application to radiation treatment of human Glioblastoma Brain Cancer. We claim that we can utilize this pharmaceutical for the targeted enhancement of radiation susceptibility and resistance in the cancerous and healthy tissues, using energetic Protons or Carbon lons and the associated Bragg Peak selected energy deposition for these regions. We claim that this pharmaceutical can also be utilized with other focused radiation modalities such as X-Rays or electron beams, but not as effectively. We specifically select the DR1709 Deinococcus Radiodurans gene for this application and the DrRRA(DR2418) Deinococcus Radiodurans gene for this application. We do not exclude other Deinococcus Radiodurans genes, and DNA/RNA genetic as well as chemical repair systems not known at this time to this inventor currently or in the future. We do not exclude genes of other micro-organisms that survive under extreme conditions. However, only the application of radiation resistant micro-organism genetic material to create a pharmaceutical for the control of radiation resistance and susceptibility is being patented with a specific organism and gene set as the basis of this Patent (i.e., Deinococcus Radiodurans) and the application to the radiation treatment of human Glioblastoma Brain Cancer.

### FEDERALLY SPONSORED RESEARCH AND DEVELOPMENT

Waiver of Government Rights on Dec. 11, 2008 from the US Department of Energy to Imagineering Technologies, Incorporated. (Refer to documentation in the Provisional Patent Application of 2024 Feb. 26 (Number 63/630,703))

### PRIOR ART

Ref 1a “The radioresistant and survival mechanisms of Deinococcus radiodurans”, Radiation Medicine and Protection,4 (2023) 70-79, Feng Liu, Nuomin Li and Yongqian Zhang

Ref 1b “Antioxidative system of Deinococcus radiodurans”, Research in Microbiology 171 (2020) 45-54, Hui-zhou Qi, Wu-zhou Wang, Jun-yan He, Yun Ma, Fang-zhu Xiao, Shu-ya He

Ref 1c “The radioresistance analysis of the Deinococcus radioduran0s gene DR1709 in human bone marrow cell line KG1a”, African Journal of Microbiology Research Vol. 6(1), pp. 40-45, 9 Jan. 2012, Shu Haiyan and Tian Baoming

### BACKGROUND OF THE INVENTION

There exists in NATURE a number of microscopic organisms that exhibit survival traits under extreme conditions. Several exhibit survival traits under extreme radiation fields. We believe that this survival trait is genetically based, per PRIOR ART above, where we have specifically selected the Deinococcus Radiodurans DNA/RNA genetic repair system for the application to radiation treatment of human Glioblastoma Brain Cancer. We believe that these genetic traits can be utilized for the targeted enhancement of radiation susceptibility and resistance and can be encapsulated in a pharmaceutical.

Energetic Protons and Carbon lons are Ideally suited for Glioblastoma Brain Cancer Treatment because of the localized Bragg Peak energy deposition behavior in human cells and H2O and can target the Cancer. (Known Physics Fact in Oncology).

We believe that a delivery technology of the relevant genetic materials to the Cancer and healthy tissue can be developed (subject of future patents) and that a number of methods are already known but not applied to the system under the current patent submission and consideration.

### SUMMARY OF THE INVENTION

A pharmaceutical selectively utilizing the Deinococcus Radiodurans genes, and DNA/RNA genetic as well as chemical repair system, for the application to radiation treatment of human Glioblastoma Brain Cancer. Utilizing this pharmaceutical for the targeted enhancement of radiation susceptibility and resistance in the cancerous and healthy tissues, using energetic Protons or Carbon lons and associated Bragg Peak selected energy deposition for these regions. Also can be utilized with other focused radiation modalities such as X-Rays or electron beams, but not as effectively.

We specifically select the DR1709 Deinococcus Radiodurans gene for this application;
and the DrRRA (DR2418) Deinococcus Radiodurans gene for this application.

We do not exclude other Deinococcus Radiodurans genes, and DNA/RNA genetic as well as chemical repair systems not known at this time to this inventor currently or in the future.

### BRIEF DESCRIPTION OF DRAWING(S)

FIG. 1: Illustration of enhanced radiation susceptibility due to the Radiodurans-Suscepta-Humana pharmaceutical genetic material transfer to a cancer cell's DNA or RNA and enhanced cancer cell death under irradiation.

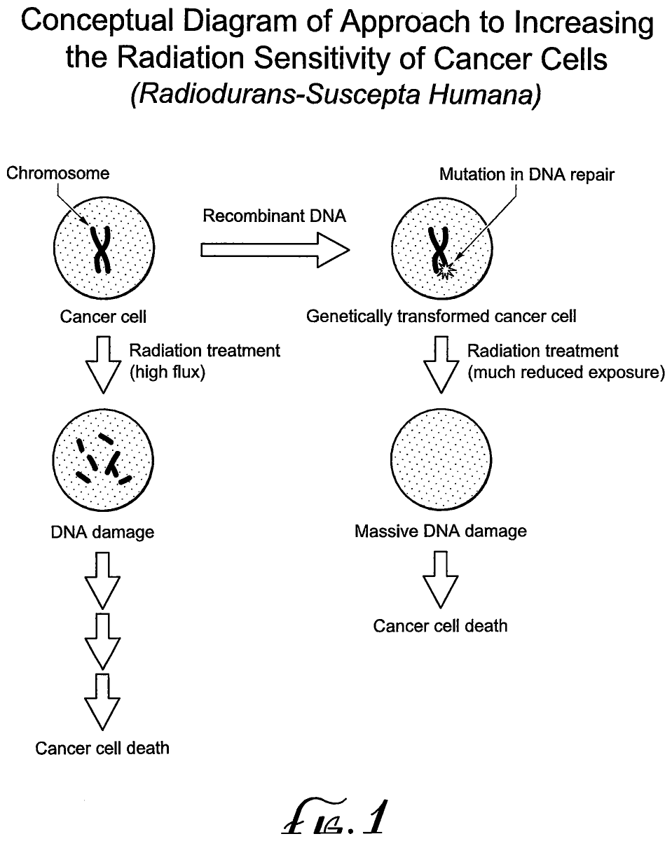

### DETAILED DESCRIPTION OF THE INVENTION

It is known that certain micro-organisms exits in nature under extreme conditions. Some survive under very large radiation fields. It has been shown that such traits are genetically based and can be transferred to other organisms and human cells (refer to the PRIOR ART section) for the micro-organism Deinococcus Radiodurans. We claim as our invention a pharmaceutical selectively utilizing the Deinococcus Radiodurans genes, and DNA/RNA genetic as well as chemical repair system, for the application to radiation treatment of human Glioblastoma Brain Cancer. We claim that we can utilize this pharmaceutical for the targeted enhancement of radiation susceptibility and resistance in the cancerous and healthy tissues, using energetic Protons or Carbon lons and the associated Bragg Peak selected energy deposition for these regions. We claim that this pharmaceutical can also be utilized with other focused radiation modalities such as X-Rays or electron beams, but not as effectively. (Refer to the SUMMARY OF INVENTION section for specific details and specified genes) We believe that other micro-organisms found in NATURE surviving under other extreme conditions may poses similar traits to Deinococcus Radiodurans and that similar pharmaceuticals can also be derived from them.

1. We specifically select the DR1709 Deinococcus Radiodurans gene for this application.

2. We specifically select the DrRRA (DR2418) Deinococcus Radiodurans gene for this application.

3. We do not exclude other Deinococcus Radiodurans genes, and DNA/RNA genetic as well as chemical repair systems not known at this time to this inventor currently or in the future.

4. We believe that other micro-organisms found in NATURE surviving under other extreme conditions may poses similar traits to Deinococcus Radiodurans and that similar pharmaceuticals can also be derived from them. We claim them in this Patent.

5. We believe that other micro-organisms found in NATURE surviving under other extreme conditions may poses similar traits to Deinococcus Radiodurans and that other pharmaceuticals can also be derived from them, such as new anti-biotics. We claim them in this Patent.

6. We claim that for Deinococcus Radiodurans, other pharmaceuticals can also be derived from it, such as new anti-biotics. We claim them in this Patent.
